(19) United States
(12) Patent Application Publication
(10) Pub. No.: US 2010/0130821 A1
(43) Pub. Date: May 27, 2010

(54) ENDOSCOPIC OVERTUBE

(75) Inventors: Alexander S. Rosemurgy, Tampa, FL (US); Sharona B. Ross, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 12/695,655

(22) Filed: Jan. 28, 2010

Related U.S. Application Data
(63) Continuation of application No. PCT/US2008/074445, filed on Aug. 27, 2008.
(60) Provisional application No. 60/968,192, filed on Aug. 27, 2007.

(51) Int. Cl. A61B 1/01

(52) U.S. Cl. 600/115

(57) ABSTRACT

An endoscopic guide for natural orifice transluminal endoscopic surgery. The device is a semi-flexible over-tube extending from the incisors through stomach and into the peritoneum. The over-tube has an inflatable balloon at its midpoint to occlude the esophageal lumen and prevent potential reflux of gastric content into the esophagus. The distal end is surrounded by two narrow inflatable balloons that are utilized on either side of the gastrotomy to keep the device in place with its lumen open into the peritoneum. The over-tube has a valve at the proximal opening that prevents leakage or peritoneal insufflation. The over-tube sheaths and protects the esophagus and guides instruments into the peritoneum. This device avoids re-intubation of the esophagus, promotes safe and easy access with increased efficiency and accuracy of insertion of the endoscope and other instruments, minimizes risks of perforation or other mucosal injuries, and allows secure control of the gastrotomy.

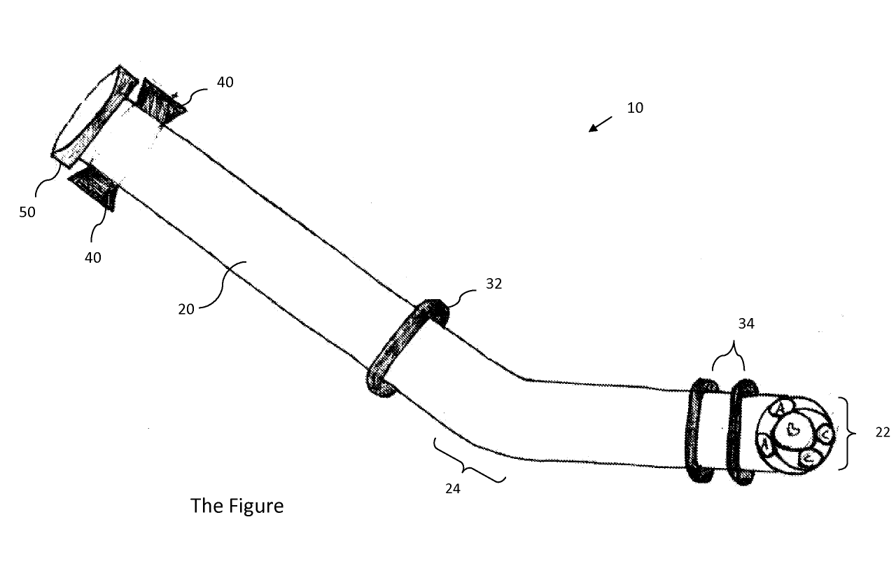

### CROSS REFERENCE TO RELATED APPLICATIONS

This application is a continuation of prior filed International Application Serial No. PCT/US2008/074445 filed Aug. 27, 2008, which claims priority to U.S. Provisional Patent Application 60/968,192 filed Aug. 27, 2007, the contents of which are herein incorporated by reference.

### FIELD OF INVENTION

This invention relates to surgical devices. More specifically, this invention relates to endoscopic guides for natural orifice transluminal endoscopic surgery.

### BACKGROUND OF THE INVENTION

Natural Orifice Transluminal Endoscopic Surgery (N.O.T.E.S. or NOTES) is an innovative surgical method that is currently under development. The NOTES surgical technique enables abdominal operations to be performed with an endo scope passed through a natural orifice, such as the mouth, then through an internal incision, such as through the stomach in the case of a procedure entering through the mouth. The procedure therefore utilizes an internal incision, thus avoiding external incisions and associated scarring. NOTES allows for minimally invasive surgery by eliminating abdominal incisions. Potential advantages of NOTES include avoidance of the potential complications of transabdominal wound infections, better postoperative pulmonary and diaphragmantic function, the potential for “scarless” abdominal surgery, lower anesthesia requirements, faster recovery and shorter hospital stays.

While endoscopic guide tubes are generally known in the art, the guides are not adapted for NOTES surgery and are generally deficient for the NOTES procedures in that they fail to provide secure transluminal access to the peritoneum. The present invention solves this shortcoming, and others, as will become apparent.

### SUMMARY OF INVENTION

An endoscopic guide for natural orifice transluminal endoscopic surgery. This invention addresses a variety of problems associated with endoscopic upper gastrointestinal access into and through the stomach anterior wall. The device is a semi-flexible over-tube extending from the incisors through the esophagus and stomach and out into the peritoneum. It has a bite block for insertion and retention in the mouth of the patient. The over-tube sheaths and protects the esophagus and guides an endoscope as well as various other instruments, through the stomach and out via the anterior wall into the peritoneum. This device avoids re-intubation of the esophagus, promotes safe and easy access with increased efficiency and accuracy of insertion of the endoscope and other instruments, minimizes risks of perforation or other mucosal injuries, and allows secure control of the gastrotomy. An overtube that extends all the way from mouth to peritoneal cavity will prevent these kind of risks. The device avoids the loss of pneumoperitoneum during the operation. Re-insertions of instruments and endoscopes into the peritoneal cavity may lead to loss of anterior gastric wall access which in turn would lead to loss of pneumoperitoneum. The device further avoids the loss of anterior gastric wall access into the peritoneum. Re-insertions of instruments and endoscope into the peritoneal cavity may lead to loss of anterior gastric wall access which in turn would increase the operative time that is spent on re-establishing gastric access This can be more difficult to achieve because of the already existing gastrotomy or gastric wall defect which prevents intraluminal gastric insufflation that allows to identify the previous opening and reinsert instruments and scope.

For the patient, the device decreases the total physician and support staff time spent on any given procedure resulting in a net benefit to the patient's health and overall prognosis as a result of a decrease in potential procedure complications and reduced time under anesthesia. To the institution and the clinician, the device will result in decrease in the overall procedure time, which, in turn, reduces the potential for patient complications and, by implication, reduces recovery time and those costs associated thereof.

In a first aspect the present invention provides an endoscopic guide tube. The guide tube includes a semi-flexible hollow cylinder having a distal and proximal end, a first expandable balloon affixed peripherally about the semi-flexible hollow cylinder and positioned on the hollow cylinder so as to occlude the esophageal lumen upon inflation of the first expandable balloon, a second and third expandable balloons affixed peripherally about the flexible hollow cylinder adjacent to the distal end of the semi-flexible hollow cylinder, the second and third expandable balloons securing the semi-flexible hollow cylinder upon inflation of the balloons thereby providing direct access to the peritoneum and allowing the insertion, manipulation and removal of instruments during a procedure, a bite block adjacent to the proximal end of the semi-flexible hollow cylinder and a valve at the proximal end of the flexible hollow cylinder. The valve allows for the sealing of the lumen of the cylinder from the external environment thereby preventing leaking or insufflation and allowing access to the flexible hollow cylinder for insertion, removal and manipulation of instruments during a procedure. The cylinder of the guide is to be inserted into a body cavity to guide the distal end portion of the flexible tube of an endoscope into the body cavity.

In certain embodiments flexible hollow cylinder has a plurality of lumens to facilitate the passage of instruments. The plurality of lumens can be sized according to the diameter of the instrument to be passaged through the lumen. The flexible hollow cylinder can be constructed of a transparent or translucent material to facilitate visualization of the structures surrounding the guide by the endoscope. Additionally, the flexible hollow cylinder can employ a curve along the length of the cylinder, the curve allowing the cylinder to adopt the anatomic shape of the passage to the stomach.

In a second aspect the present invention provides a second embodiment of an endoscopic guide tube. The guide tube includes a semi-flexible hollow cylinder having a distal and proximal end and a first and second expandable balloons affixed peripherally about the flexible hollow cylinder adjacent to the distal end of the semi-flexible hollow cylinder. The first and second expandable balloons secure the semi-flexible hollow cylinder upon inflation thereby providing direct access to the peritoneum and allowing the insertion and removal of instruments during a procedure.

In a third aspect aspect the present invention provides a third embodiment of an endoscopic guide tube. The guide tube includes a semi-flexible hollow cylinder having a distal and proximal end, a first and second expandable balloons affixed peripherally about the flexible hollow cylinder adjacent to the distal end of the semi-flexible hollow cylinder and a valve at the proximal end of the flexible hollow cylinder. The valve allows for the sealing of the lumen of the cylinder from the external environment thereby preventing leaking or insufflation and allowing access to the flexible hollow cylinder for insertion, removal and manipulation of instruments during a procedure. The first and second expandable balloons secure the semi-flexible hollow cylinder upon inflation thereby providing direct access to the peritoneum and allowing the insertion and removal of instruments during a procedure.

### BRIEF DESCRIPTION OF THE DRAWINGS

For a fuller understanding of the invention, reference should be made to the following detailed description, taken in connection with the accompanying drawing, in which:

The FIGURE is perspective view of one embodiment of the endoscopic over-tube according to the invention

### DETAILED DESCRIPTION OF THE PREFERRED EMBODIMENT

The invention is an endoscopic over-tube for receiving and guiding endoscopic instruments into the esophagus, stomach, and out into the peritoneum of a patient. An exemplary embodiment of the endoscopic overtube is presented in The FIGURE. Generally, the exemplary device is a semi-flexible over-tube 10 with a diameter large enough to allow a dual channel gastroscope and various other instruments to pass through it. The over-tube 10 extends from outside of the oral cavity of a patient, down the esophagus, through the stomach, and out into the peritoneum. over-tube 10 has a bite block 40 for insertion and retention in the mouth of the patient. At approximately 34 cm from the proximal end of the over-tube 10 an inflatable balloon 32 is used to occlude the esophageal lumen and prevent potential reflux of gastric content into the esophagus. The distal end of the over-tube 10 is surrounded by two narrow inflatable balloons 34 that are utilized on either side of the gastrotomy to keep the device (over-tube 10) in place with its lumen open into the peritoneum. The over-tube 10 has a valve 50 at the proximal opening that will prevent leakage of peritoneal insufflation.

More particularly, the exemplary over-tube 10 is an elongated cylinder 20 with a plurality of lumens 22 with openings for access to the lumens at the distal and proximal ends of the overtube 10. The cylinder 20 is preferably constructed of a semi-flexible, transparent material. By making the cylinder semi-flexible, as opposed to flexible or rigid, the device is able to offer some structure as a guide while avoiding the risk of damage to delicate structures along the path to the stomach. The device 10 of the FIGURE has a stiffened region along the cylinder 20 that protects the posterior wall of the patient's pharynx from iatrogenic injury as medical instrumentations and provides an enlarged lumen 22B having sufficient size to allow the passage of a dual channel gastroscope, as the gastroscope is inserted through the sheath and into the patient's upper gastrointestinal tract. Depending upon the size and other features of the gastroscope employed, the size of the lumen 22B for receiving and guiding the gastroscope can be tailored accordingly as one of skill in the art will appreciate. In the case of a dual channel gastroscope, the lumen 22B might have a diameter of 8.6 mm to 12.6 mm. Were one to employ a gastroscope of smaller diameter, the lumen would be adjusted proportionately. The over-tube 10 of the FIGURE also employs a curve 24 at a point along the cylinder 20 to enable the over-tube 10 to follow the anatomic shape of the path leading to the stomach. An exemplary over-tube 10 can have a length of 45.7 cm to 55.9 cm (18-22 inches) to allow it to extend from the patients mouth and down to the desired region in the stomach. As one of skill in the art will appreciate, depending upon the particular needs of the practitioner, the length and the diameter of the over-tube 10 can be tailored to meet those needs.

The over-tube 10 has a bite block 40 at the proximal end of the over-tube 10, with a flexible protective sheath in an adjacent region of the cylinder 20 that engages the bite block 40 and adapts for insertion into the esophagus, stomach, and out into the peritoneum of the patient. The over-tube 10 also has a proximal valve 50 that prevents leaks or insufflation as instruments and endoscopes are inserted, manipulated and removed within the lumens 22A-C of the over-tube 10.

The over-tube 10 has a stiffened region that protects the posterior wall of the patient's pharynx from iatrogenic injury as medical instrumentations and a dual channel gastroscope are inserted through the sheath and into the patient's upper gastrointestinal tract. An inflatable balloon 32 is present at approximately 34 cm from the proximal end of the over-tube 10 (or at a point roughly midway along the over-tube's length) to occlude the esophageal lumen and prevent potential reflux of gastric content into the esophagus. The distal end of the over-tube 10 is surrounded by two narrow inflatable balloons 34 that are utilized on either side of the gastrotomy to keep the device in place with its lumen open into the peritoneum and further prevent leakage of insufflation. In other words, each of the balloons 34 can be positioned on either side of the incision through the stomach into the peritioneum, thereby effectively securing the device in its location. This enables a user to gain access to the peritioneum, with the opportunity to manipulate instruments and gastroscopes without risking loss of access to the passage. In contrast, loss of access can lead to the requirement to insufflate to regain access. Insufflation can be difficult to achieve where there exists an incision into the peritoneum and the time lost can be significant. The exemplary over-tube 10 has three openings/lumens 22 (or formats of openings). One lumen opening 22B has an outer diameter ranging from 8.6 mm for single lumen gastroscope to 12.6 mm dual channel gastroscope (instrument channel sizes 2.8 and 3.8 mm). The over-tube also contains 2 additional instrument channels (22A and 22C) (size 2.8 mm and 3.8 mm in the outer diameter). The exemplary over-tube 10 length ranges from 45.7 cm to 55.9 cm.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. An endoscopic guide tube comprising:
a semi-flexible hollow cylinder having a distal and proximal end, the cylinder to be inserted into a body cavity to guide the distal end portion of the flexible tube of an endoscope into the body cavity;
a first expandable balloon affixed peripherally about the semi-flexible hollow cylinder and positioned on the hollow cylinder so as to occlude the esophageal lumen upon inflation of the first expandable balloon;
a second and third expandable balloons affixed peripherally about the flexible hollow cylinder adjacent to the distal end of the semi-flexible hollow cylinder, the second and third expandable balloons securing the semi-flexible hollow cylinder upon inflation of the balloons thereby providing direct access to the peritoneum and allowing the insertion, manipulation and removal of instruments during a procedure;
a bite block adjacent to the proximal end of the semi-flexible hollow cylinder; and
a valve at the proximal end of the flexible hollow cylinder, the valve sealing the lumen of the cylinder from the external environment thereby preventing leaking or insufflation and allowing access to the flexible hollow cylinder for insertion, removal and manipulation of instruments during a procedure.

2. The endoscopic guide tube of claim 1 wherein the flexible hollow cylinder comprises a plurality of lumens to facilitate the passage of instruments.

3. The endoscopic guide tube of claim 2 wherein two or more of the plurality of lumens are sized according to the diameter of the instrument to be passaged through the lumen.

4. The endoscopic guide tube of claim 1 wherein the flexible hollow cylinder is constructed of a transparent or translucent material to facilitate visualization of the structures surrounding the guide by the endoscope.

5. The endoscopic guide tube of claim 1 wherein the flexible hollow cylinder comprises a curve along the length of the cylinder, the curve allowing the cylinder to adopt the anatomic shape of the passage to the stomach.

6. An endoscopic guide tube comprising:
a semi-flexible hollow cylinder having a distal and proximal end to be inserted into a body cavity to guide the distal end portion of the flexible tube of an endoscope into the body cavity; and
a first and second expandable balloons affixed peripherally about the flexible hollow cylinder adjacent to the distal end of the semi-flexible hollow cylinder, the first and second expandable balloons securing the semi-flexible hollow cylinder upon inflation thereby providing direct access to the peritoneum and allowing the insertion and removal of instruments during a procedure.

7. The endoscopic guide tube according to claim 6 further comprising a valve at the proximal end of the flexible hollow cylinder, the valve sealing the lumen of the cylinder from the external environment thereby preventing leaking or insufflation and insertion, removal and manipulation of instruments during a procedure.

8. The endoscopic guide tube according to claim 6 further comprising a bite block adjacent to the proximal end of the flexible hollow cylinder.

9. The endoscopic guide tube according to claim 6 further comprising a third expandable balloon affixed peripherally about the semi-flexible hollow cylinder and positioned on the hollow cylinder so as to occlude the esophageal lumen upon inflation of the third expandable balloon.

10. The endoscopic guide tube of claim 6 wherein the flexible hollow cylinder comprises a plurality of lumens to facilitate the passage of instruments.

11. The endoscopic guide tube of claim 10 wherein two or more of the plurality of lumens have a diameter sized according to the diameter of the instrument to be passaged through the lumen.

12. The endoscopic guide tube of claim 6 wherein the flexible hollow cylinder is constructed of a transparent or translucent material to facilitate visualization of the structures surrounding the guide by the endoscope.

13. The endoscopic guide tube of claim 6 wherein the flexible hollow cylinder comprises a curve along the length of the cylinder, the curve allowing the cylinder to adopt the anatomic shape of the passage to the stomach.

14. An endoscopic guide tube comprising:
a semi-flexible hollow cylinder having a distal and proximal end to be inserted into a body cavity to guide the distal end portion of the flexible tube of an endoscope into the body cavity;
a first and second expandable balloons affixed peripherally about the flexible hollow cylinder adjacent to the distal end of the semi-flexible hollow cylinder, the first and second expandable balloons securing the semi-flexible hollow cylinder upon inflation thereby providing direct access to the peritoneum and allowing the insertion and removal of instruments during a procedure; and
a valve at the proximal end of the flexible hollow cylinder, the valve sealing the lumen of the cylinder from the external environment thereby preventing leaking or insufflation and allowing access to the flexible hollow cylinder for insertion, removal and manipulation of instruments during a procedure.

15. The endoscopic guide tube according to claim 14 further comprising a bite block adjacent to the proximal end of the flexible hollow cylinder.

16. The endoscopic guide tube according to claim 14 further comprising a third expandable balloon affixed peripherally about the semi-flexible hollow cylinder and positioned on the hollow cylinder so as to occlude the esophageal lumen upon inflation of the third expandable balloon.

17. The endoscopic guide tube of claim 14 wherein the flexible hollow cylinder comprises a plurality of lumens to facilitate the passage of instruments.

18. The endoscopic guide tube of claim 17 wherein two or more of the plurality of lumens have a diameter sized according to the diameter of the instrument to be passaged through the lumen.

19. The endoscopic guide tube of claim 14 wherein the flexible hollow cylinder is constructed of a transparent or translucent material to facilitate visualization of the structures surrounding the guide by the endoscope.

20. The endoscopic guide tube of claim 14 wherein the flexible hollow cylinder comprises a curve along the length of the cylinder, the curve allowing the cylinder to adopt the anatomic shape of the passage to the stomach.
